(19) United States
(12) Patent Application Publication
(10) Pub. No.: US 2005/0159758 A1
(43) Pub. Date: Jul. 21, 2005

(54) Ophthalmic irrigation-aspiration system

(76) Inventor: Lawrence Laks, Bellevue, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/980,898

(22) Filed: Jul. 26, 2004

Related U.S. Application Data
(60) Provisional application No. 60/490,164, filed on Jul. 25, 2003.

(51) Int. Cl. A61F 9/00

(52) U.S. Cl. 606/107; 604/30

(57) ABSTRACT

An irrigating component provides a flow of irrigation liquid into a lens capsule or chamber during eye surgery while a separate aspiration component withdraws fluid from the capsule or chamber. The irrigating component is designed to permit a flow rate that at least balances the flow rate of the aspiration component.

### CROSS-REFERENCE TO RELATED APPLICATION

This application claims the benefit of U.S. Provisional Application No. 60/490,164, filed on Jul. 25, 2003.

### FIELD OF THE INVENTION

The present invention relates to an ophthalmic surgical instrument system having one component that provides a flow of irrigating liquid into a lens capsule during use and a separate component for aspiration. The two components have tips that are inserted through separate incisions or tunnels, preferably a substantial distance apart.

### BACKGROUND OF THE INVENTION

Cataract surgery uses phaco emulsifiers, choppers, irrigators, and aspirators to emulsify and/or chop a clouded natural lens and clean the lens capsule or chamber in preparation for insertion of an artificial lens.

### SUMMARY OF THE INVENTION

The present invention provides an ophthalmic irrigation instrument, which in combination with an ophthalmic aspiration instrument, generates irrigation and aspiration flow rates sufficient to prevent collapse of a lens capsule or chamber during a “bimanual” procedure.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a top perspective of an ophthalmic irrigation handpiece;

FIG. 2 is a top plan thereof;

FIG. 3 is a side elevation thereof; and

FIG. 4 is a longitudinal section thereof.

FIG. 5 is an enlarged, somewhat diagrammatic, top perspective of an irrigating chopper tip, in a preliminary stage of manufacture, usable with the handpiece of FIGS. 1-4;

FIG. 6 is a side elevation thereof;

FIG. 7 is a longitudinal section thereof;

FIG. 8 is a front end elevation thereof; and

FIG. 9 is a rear end elevation thereof.

FIG. 10 is an enlarged, somewhat diagrammatic, top perspective of another irrigating chopper tip usable with the handpiece of FIGS. 1-4;

FIG. 11 is a side elevation thereof;

FIG. 12 is a further enlarged, fragmentary detail of the distal tip portion thereof; and

FIG. 13 is an enlarged, fragmentary section of the distal tip portion thereof.

FIG. 14 is an enlarged, somewhat diagrammatic, top perspective of another irrigating chopper tip usable with the handpiece of FIGS. 1-4;

FIG. 15 is a side elevation thereof;

FIG. 16 is a further enlarged, fragmentary detail of the distal tip portion thereof; and

FIG. 17 is a detail view taken from B-B of FIG. 15.

FIG. 18 is an enlarged, somewhat diagrammatic, top perspective of another irrigating chopper tip usable with the handpiece of FIGS. 1-4;

FIG. 19 is a side elevation thereof;

FIG. 20 is a further enlarged, fragmentary detail of the distal tip portion thereof; and

FIG. 21 is a detail view taken from B-B of FIG. 19.

FIG. 22 is an enlarged, somewhat diagrammatic, top perspective of another irrigating chopper tip usable with the handpiece of FIGS. 1-4;

FIG. 23 is a side elevation thereof;

FIG. 24 is an enlarged, fragmentary detail of the distal tip portion thereof;

FIG. 25 is a detail view taken from B-B of FIG. 23;

FIG. 26 is a detail view taken from C-C of FIG. 25; and

FIG. 27 is a detail view taken from D-D of FIG. 26.

FIG. 28 is an enlarged, somewhat diagrammatic, top perspective of another irrigating chopper tip usable with the handpiece of FIGS. 1-4;

FIG. 29 is a side elevation thereof;

FIG. 30 is an enlarged, fragmentary detail of the distal tip portion thereof;

FIG. 31 is a detail view taken from B-B of FIG. 29; and

FIG. 32 is a detail view taken from C-C of FIG. 29.

FIG. 33 is an enlarged, somewhat diagrammatic, top perspective of another irrigating tip usable with the handpiece of FIGS. 1-4; and

FIG. 34 is a side elevation thereof.

FIG. 35 is an enlarged, somewhat diagrammatic, top perspective of another irrigating tip usable with the handpiece of FIGS. 1-4; and

FIG. 36 is a side elevation thereof.

FIG. 37 is a top perspective of an ophthalmic aspiration handpiece;

FIG. 38 is a top plan thereof;

FIG. 39 is a side elevation thereof; and

FIG. 40 is a longitudinal section thereof.

FIG. 41 is an enlarged, somewhat diagrammatic, top perspective of an aspiration tip usable with the handpiece of FIGS. 37-40; and

FIG. 42 is a side elevation thereof.

FIG. 43 is an enlarged, somewhat diagrammatic, top perspective of another aspiration tip usable with the handpiece of FIGS. 37-40; and

FIG. 44 is a side elevation thereof.

FIG. 45 is a diagrammatic perspective view of a fragment of an eye and instruments used in the system of the present invention during cataract removal.

### DETAILED DESCRIPTION OF THE PREFERRED EMBODIMENT

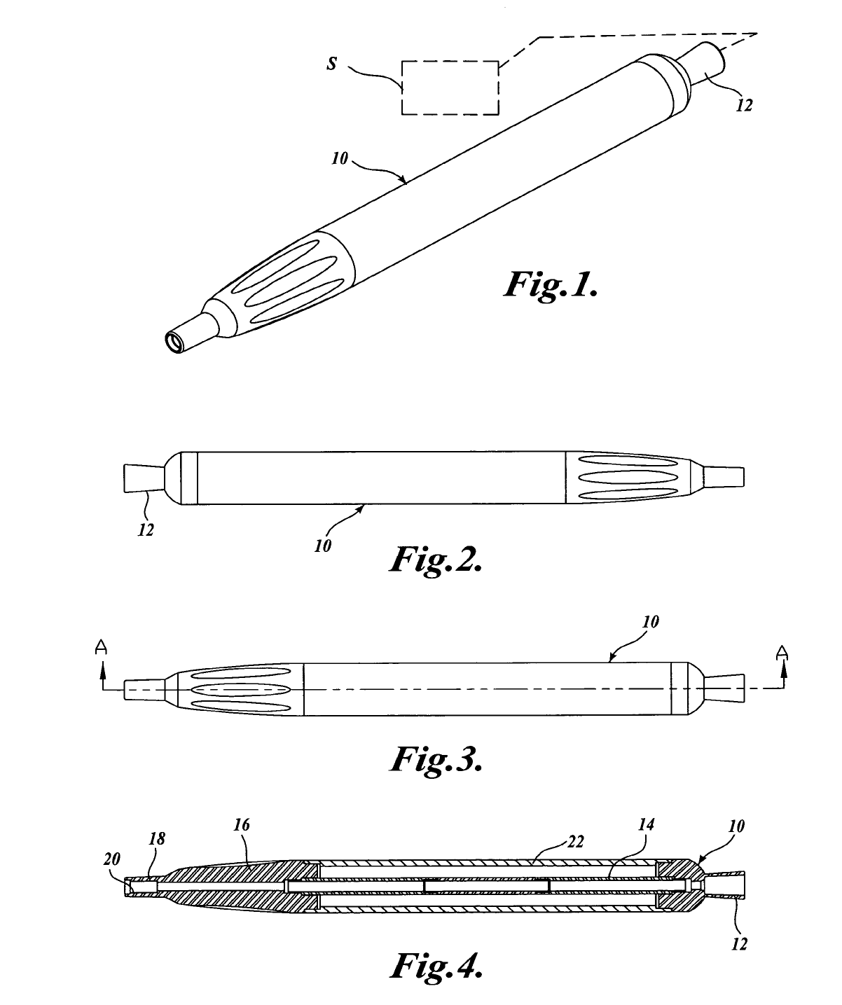

FIGS. 1-4 illustrate a representative irrigator handpiece 10 used in the system of the present invention. The proximate end of the handpiece has a coupling 12 for connection to a source S of irrigating liquid under pressure. Coupling 12 opens through the proximate end portion of the handpiece to an inner conduit 14. Conduit 14 leads to the distal end portion 16 of the handpiece, which may be contoured for convenient grasping. The distal end 18 forms an internally threaded socket 20. The components of the handpiece can be press-fitted or otherwise secured together. The long central portion of the bandpiece can be formed by a tubular shell and the conduit 14 may be formed of several sections joined end to end. Preferably, the long central channel or bore is open and unobstructed for free flow of irrigating liquid therethrough.

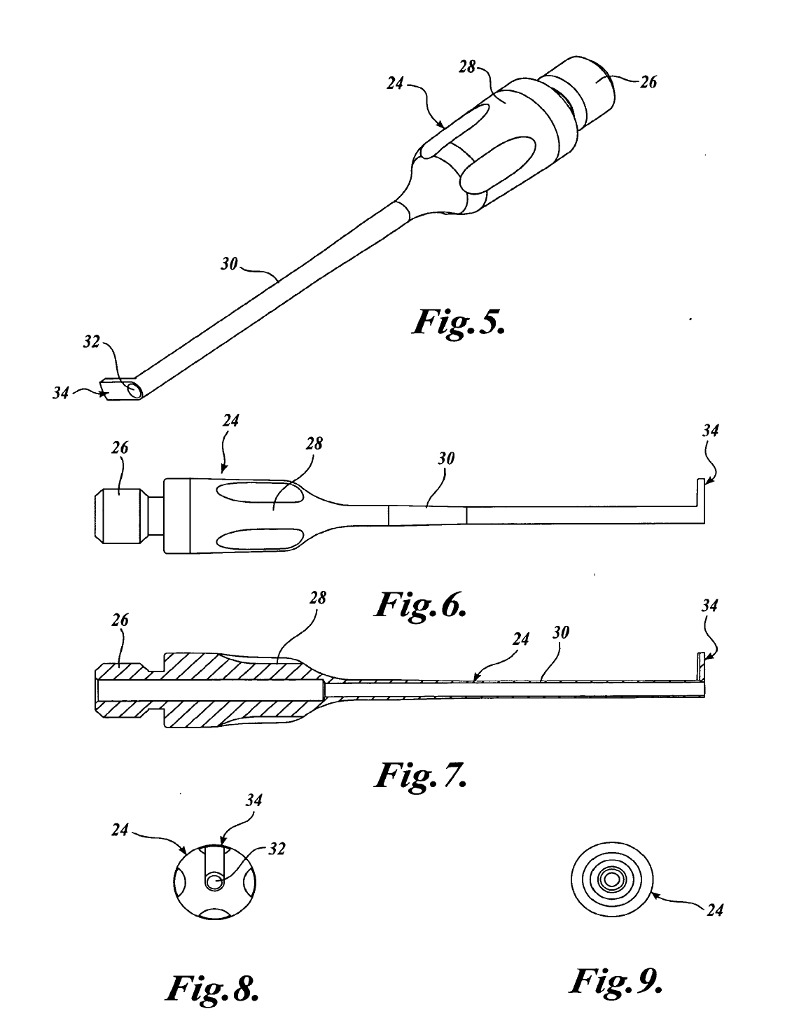

FIGS. 5-9 show an irrigating chopper tip 24 usable in the system of the present invention, at an intermediate stage of manufacture. Preferably, the tip 24 is formed of a rigid, machineable metal, such as titanium or a suitable alloy. Tip 24 has a proximate stem 26 that is externally threaded to mate with the internal threads of the distal end of the handpiece. Stem 26 leads to a hub 28, which may be configured for grasping by a user or suitable wrench or other tool for attaching the tip to the handpiece. Hub 28 also may form a proximate shoulder that limits insertion of the stem into the threaded bore of the handpiece. A long, narrow tube 30 extends distally from the hub 28, with a distal end opening 32. The long bore or channel of the tip 24 is open and unobstructed throughout its entire length, permitting a free flow of irrigating liquid therethrough. At the distal end of the tip, a chopper component 34 projects substantially radially outward from the end of the tube. In FIGS. 5-9, the chopper is illustrated as being of rectangular cross-section. The chopper 34 can be sharpened by machining to any one of several different chopping configurations. For example, the radially outer end of the chopper 34 can be sharpened for a “tomahawk” chopping action and/or the side, trailing, or leading edges can be sharpened to achieve a desired chopping configuration. Since the entire tip 24 is formed in one piece, with all aspects integral, there are no crimps, bends, adhesive, welding beads, etc., to interfere with free flow of irrigation liquid through the tip, or with chopping effectiveness of a desired distal end configuration. The threaded stem also is configured to achieve a smooth flow of liquid when mated to the associated handpiece, preferably with the proximate end abutting or nearly abutting the base of the handpiece socket.

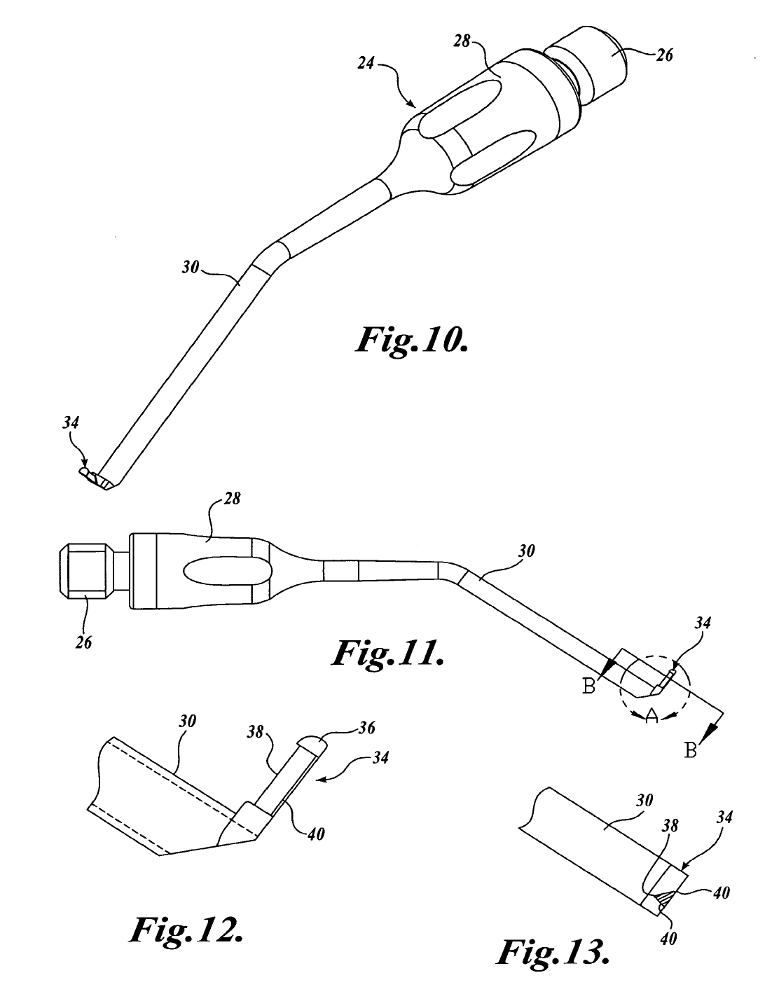

In the embodiment of FIGS. 10-13, the configuration of the stem 26 and hub 28 are identical to the embodiment previously described, but the long distal tube 30 (formed integral) is bent at a moderate angle. The chopper projection 34 is machined with a blunt, almost hemispherical, radially outer end 36, whereas the proximate edge 38 and transverse edges 40 of the chopper are sharpened. The distal end of the irrigator tube 30 is beveled as illustrated in these views.

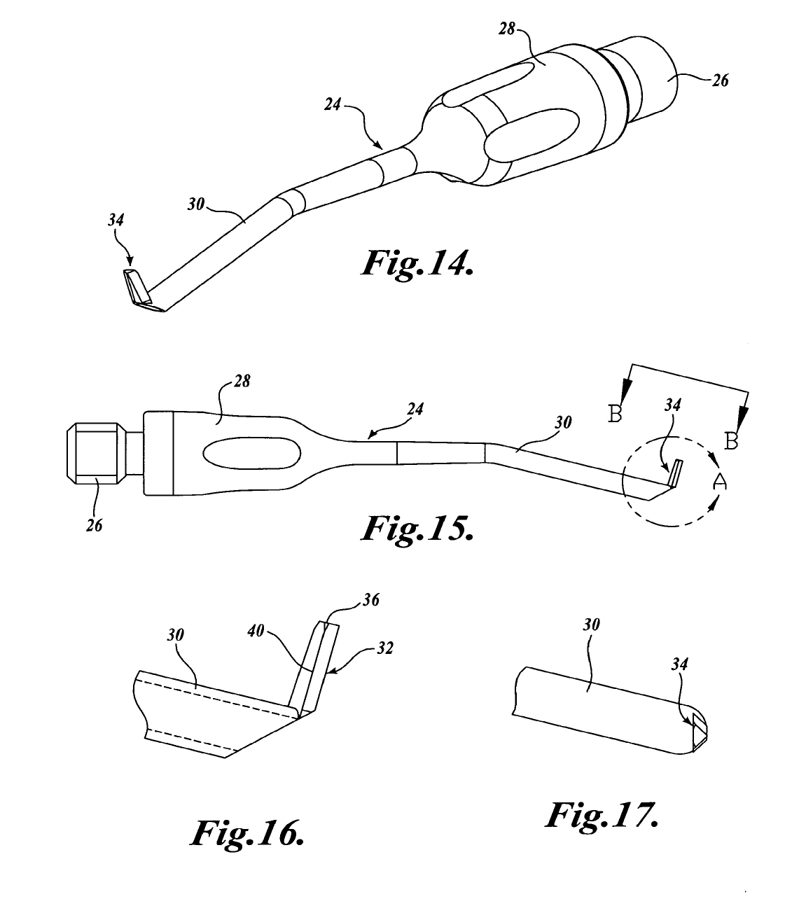

The embodiment of FIGS. 14-17 is essentially the same as the embodiment of FIGS. 10-13 except for the more moderate bend of the tube 30 and the sharpening of the chopper 34. The side edges 40 are sharpened to a somewhat greater degree than the radially outer tip 36, with some sharpening of the leading or distal edge as well as the trailing or proximate edge.

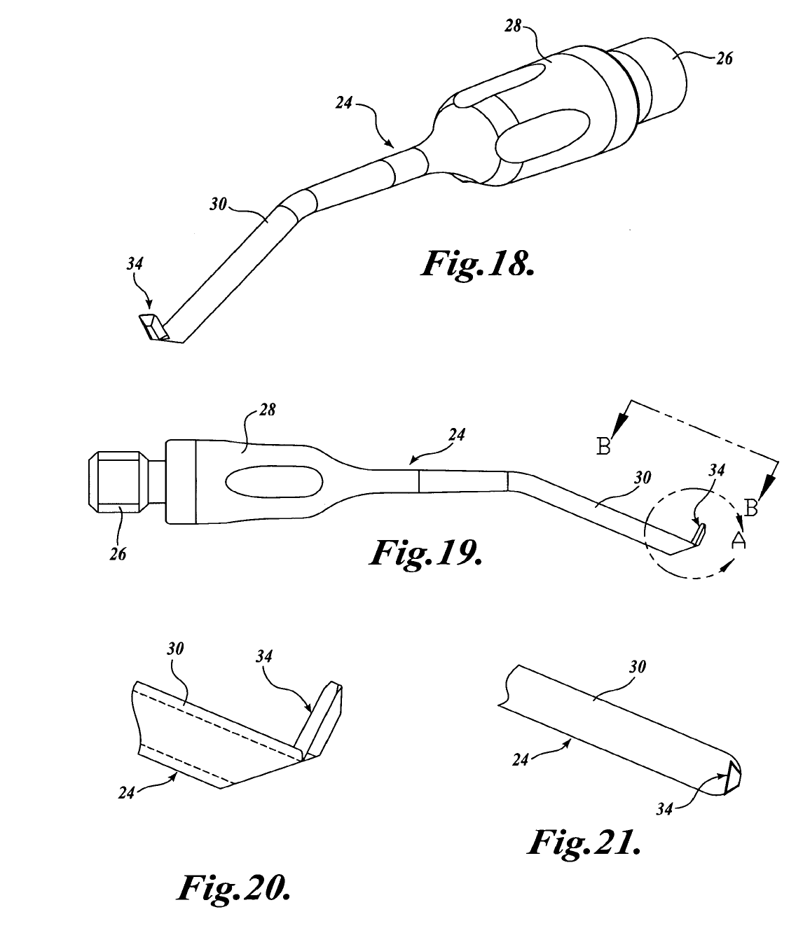

Similarly, the embodiment of FIGS. 18-21 is essentially the same as the embodiment of FIGS. 14-17, except for a modified sharpening of the chopper 34, which is similar but not identical.

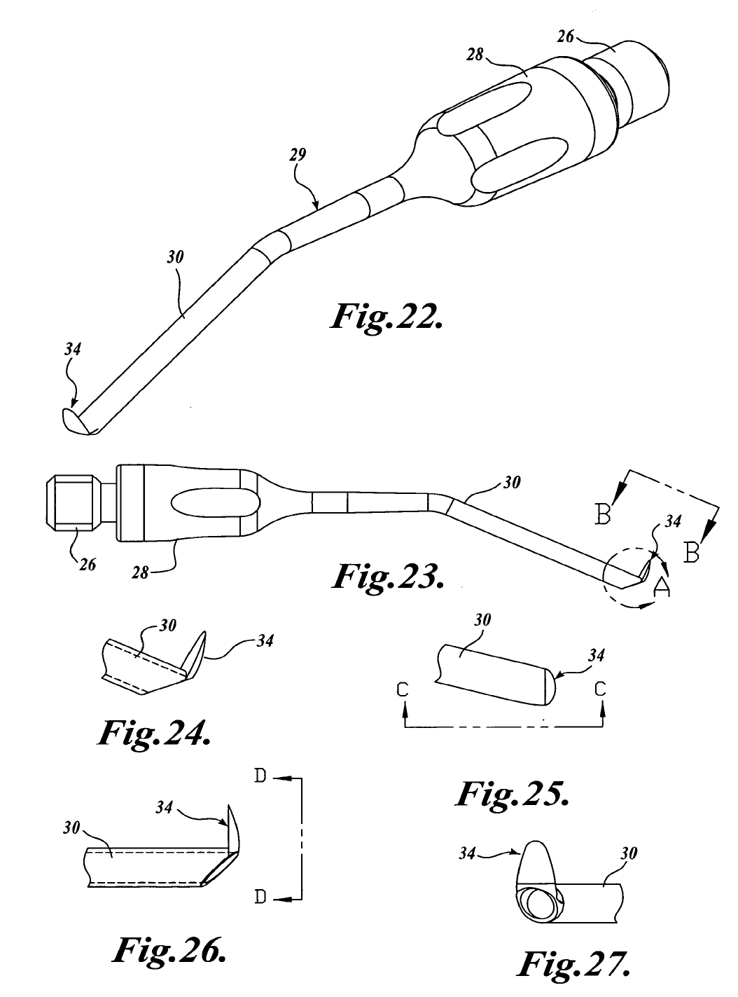

In the embodiment of FIG. 22-27, the chopper 34 has a blunt, rounded distal face, whereas the side and radially outer edges are sharpened for an effective chopping action.

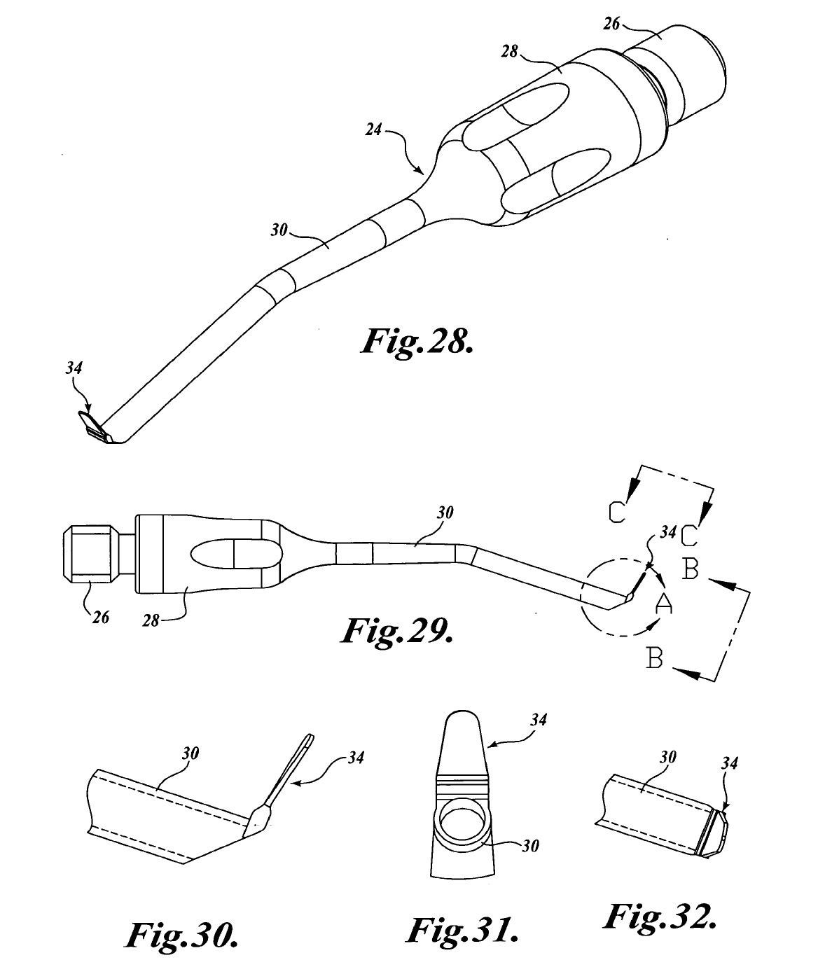

In the embodiment of FIGS. 28-32, the chopper 34 is machined to a thin blade-like configuration with essentially flat proximate and distal surfaces.

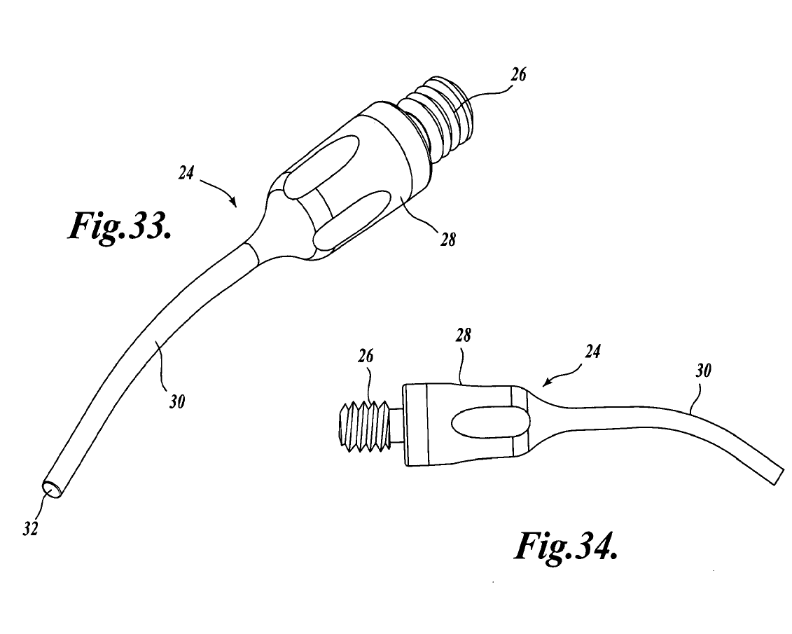

In the embodiment of FIGS. 33 and 34, the irrigation tip 24 has the same stem 26 and hub 28 as the previously described embodiments, with a curved, long, narrow tube 30 extending distally from the hub. Tube 30 has a distal end opening 32 for expelling irrigation liquid, but no integral, radially projecting chopper.

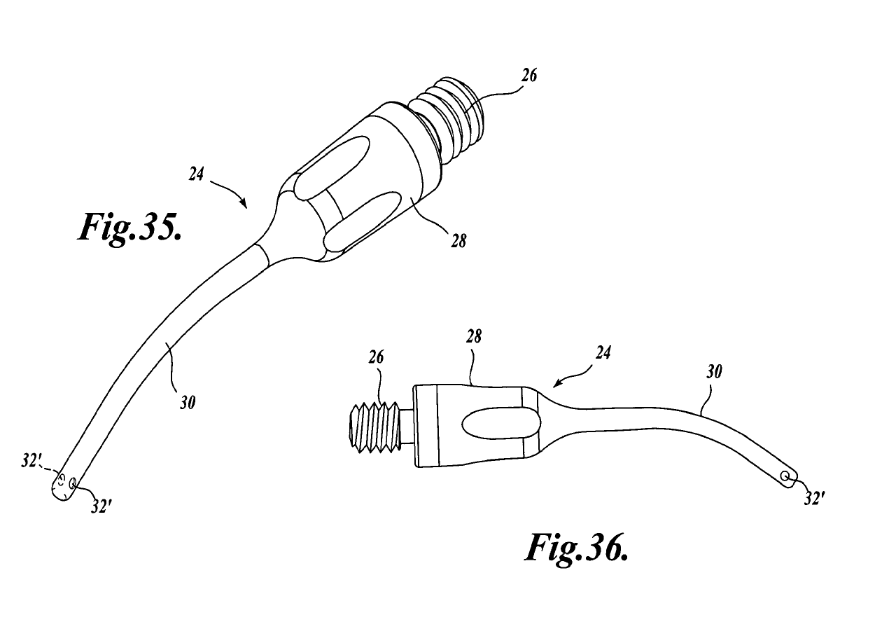

In the embodiment of FIGS. 35 and 36, the irrigator 24 again has the same threaded stem 26, hub 28, and narrow tube 30. However, the distal end of the tube is closed, and a port 32′ is provided at each side for expelling irrigation liquid.

In the preferred embodiment the tube 30 is formed integrally with the hub 28 and stem 26, but in alternative embodiments the parts could be formed separately and, for example, press fitted together, so long as there is no interference with flow through the bore of the irrigation tip. Known designs have crimps or welds or other features or designs at one or more locations, for which may limit the flow rate of irrigation fluid from the tip.

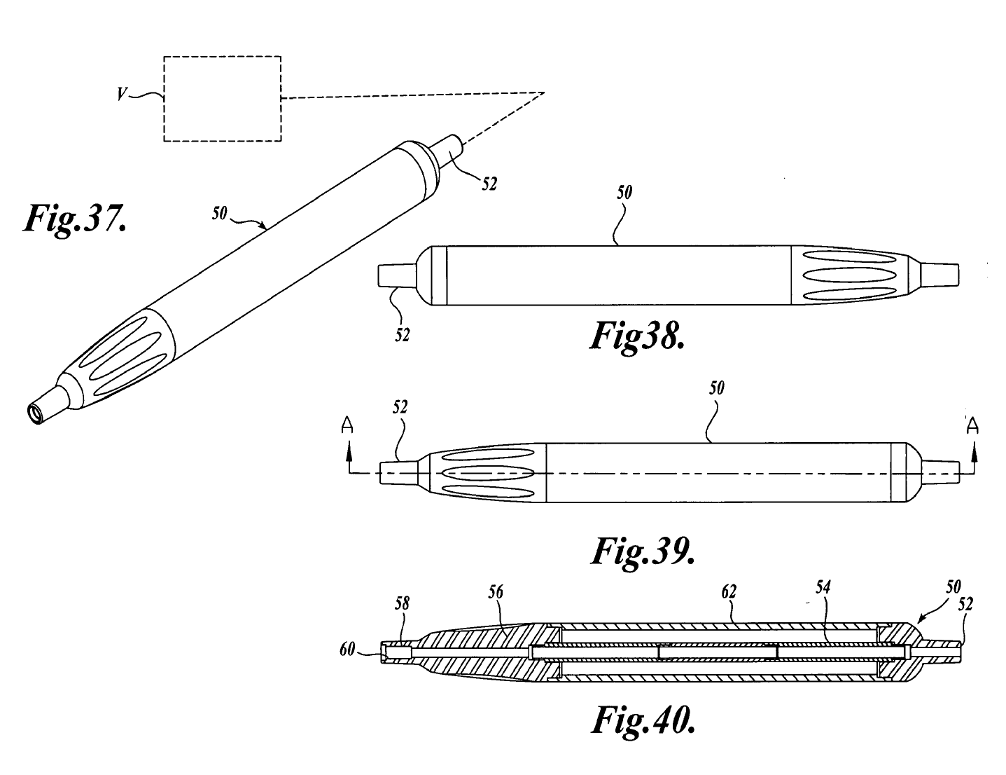

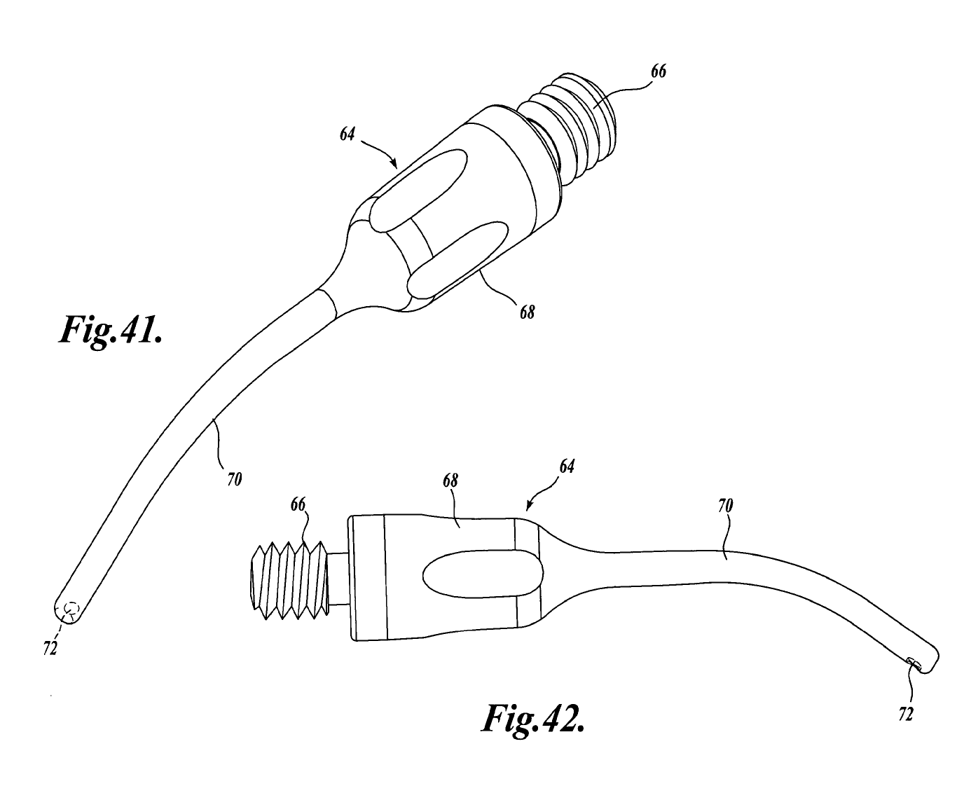

FIGS. 37-40 illustrate a representative aspirator handpiece 50 used in the system of the present invention. The proximate end of the handpiece has a coupling 52 for connection to a source V of reduced pressure or vacuum. Coupling 52 opens through the proximate end portion of the handpiece to an inner conduit 54. Conduit 54 leads to the distal end portion 56 of the handpiece, which may be contoured for convenient grasping. The distal end 58 forms an internally threaded socket 20, identical to the socket of the irrigator handpiece discussed above. The long central portion of the handpiece 50 can be formed by a tubular shell, and the conduit 54 may be formed of several sections adjoined end to end. Preferably the long central channel or bore is open and essentially unobstructed for free flow of aspirated liquid therethrough.

FIGS. 41 and 42 show an aspiration tip 64 usable in the system of the present invention. Preferably the tip 64 is formed of a rigid, machineable metal, such as titanium or a suitable alloy. Tip 64 has a proximate stem 66 that is externally threaded to mate with the internal threads of the distal end of the aspiration handpiece. Stem 66 leads to a hub 68, which may be configured for grasping by a user or suitable wrench or other tool for attaching the tip to the handpiece. Hub 68 also may form a proximate shoulder that limits insertion of the stem into the threaded bore of the handpiece. A long, narrow tube 70 extends distally from the hub 68 and aspiration port 72 formed in the underside of the distal end portion of the tube.

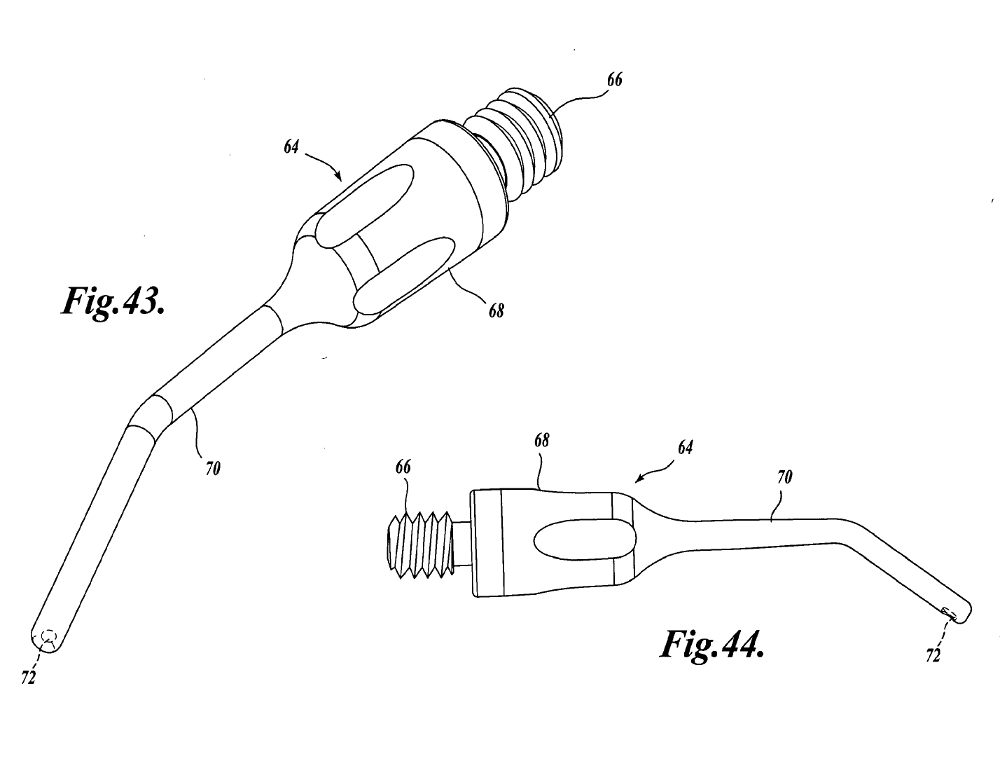

In the embodiment of FIGS. 43 and 44, the configuration of the stem 66 and hub 68 are identical to the aspiration tip embodiment previously described, but with the long distal tube 30 bent more abruptly and at a slightly different angle, as may be preferred by a user. Otherwise, the aspiration tip of FIG. 43 is identical to the aspiration tip of FIGS. 41 and 42.

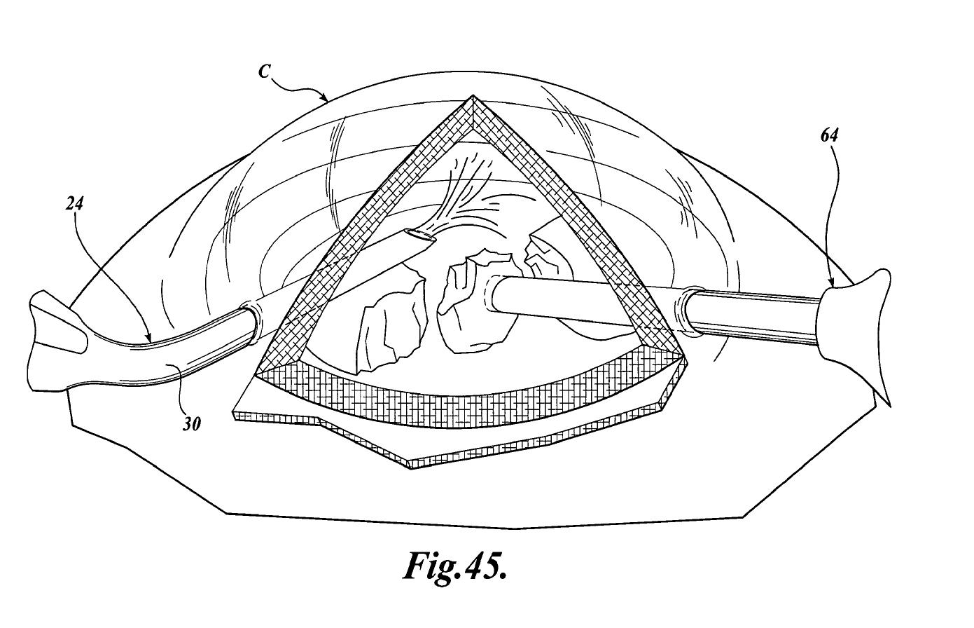

FIG. 45 shows irrigation and aspiration instruments used in the present invention and during cataract surgery, i.e., during removal of pieces of a clouded lens. One of the irrigation tips 24 is positioned with its distal tube 30 extending closely through a tunnel or slit in or adjacent to the cornea C. One of the aspiration tips 64 extends through a corresponding slit or tunnel spaced a substantial distance from the slit or tunnel for the irrigation tip. The end configuration of the aspiration tip can be as shown in FIGS. 41, 42, 43, and 44, or another configuration, such as that illustrated in FIG. 45 in which the aspiration tip has an open end which can assist in holding a piece of the clouded lens while it is chopped. In accordance with the present invention, the flow rate of irrigation liquid from the irrigating tip is balanced with the aspiration rate from the aspirator tip, to maintain adequate pressure and prevent collapse of the cornea.

While the preferred embodiment of the invention has been illustrated and described, it will be appreciated that various changes can be made therein without departing from the spirit and scope of the invention.

1. An ophthalmic irrigation-aspiration system comprising:
an aspirator component having a tip insertable through an ophthalmic slit or tunnel during eye surgery for withdrawing fluid from a lens capsule or chamber; and
an irrigator component having a tip insertable through an ophthalmic slit or tunnel during the surgery for supplying irrigating liquid at a flow rate that at least balances the aspiration rate of the aspirator component to prevent collapse of the lens capsule or chamber during the eye surgery.

2. The ophthalmic irrigation-aspiration system defined in claim 1, in which the irrigator component includes a handpiece having a distal end forming an internally threaded socket, and a separate tip component having a stem having external threads that mate with the threads of the socket.
